(19) United States
(12) Patent Application Publication
(10) Pub. No.: US 2006/0269950 A1
(43) Pub. Date: Nov. 30, 2006

(54) Method of identifying compounds that modulate interaction of androgen receptor with beta-catenin

(75) Inventors: Edward Judson Kilbourne, Hopewell, NJ (US); Thomas J. Berrodin, Collegeville, PA (US)

Correspondence Address: POTTER ANDERSON & CORROON LLP/WYETH, 1313 NORTH MARKET STREET, HERCULES PLAZA, SIXTH FLOOR, WILMINGTON, DE, 19801, US

(73) Assignee: Wyeth, Madison, NJ (US)

(21) Appl. No.: 11/435,947

(22) Filed: May 17, 2006

Related U.S. Application Data
(60) Provisional application No. 60/682,580, filed on May 19, 2005.

(51) Int. Cl. C12Q 1/68; C12Q 1/66

(52) U.S. Cl. 435/6; 435/8

(57) ABSTRACT

Methods for determining if test compounds are able to modulate the interaction between androgen receptor and β-catenin are disclosed. Methods for the determining whether a test compound selectively modulates an androgen receptor signaling pathway over a β-catenin-Wnt signaling pathway or a β-catenin-Wnt signaling pathway over an androgen receptor signaling pathway are also disclosed.

### CROSS-REFERENCE TO RELATED APPLICATION

This application claims priority from U.S. Provisional Application No. 60/682,580, filed May 19, 2005, which is incorporated by reference herein in its entirety.

### FIELD OF THE INVENTION

The present invention relates to an assay for identification of compounds that modulate the androgen-dependent interaction between androgen receptor (AR) and β-catenin. This invention particularly relates to the identification of molecules which may be able to disrupt the interaction of androgen receptor and β-catenin and thereby specifically remove the effect of β-catenin on AR signaling or remove the effect of AR on β-catenin and Wnt signaling.

### BACKGROUND OF INVENTION

Traditionally, nuclear/steroid receptor binding ligands are identified by screening for the ability of test compounds to affect the transcription of genes containing consensus nuclear receptor DNA elements responsive to that nuclear/steroid receptor. However, some steroid receptors also affect, and are affected by, non-steroidal signaling pathways in addition to their classical steroidal transcriptional control mechanisms. In many instances, it would be useful to have a means to identify specific compounds that are selectively effective in modulating only the non-steroidal signaling pathway associated with such receptor or compounds that are selectively effective in modulating only the transcriptional activities of the receptor on genes traditionally responsive to such receptor. Compounds with such selectivity would have potential pharmaceutical utility in situations where modulation of the non-steroidal pathway is desired but inhibition of classical steroid receptor mediated transcription is not desired, or vice versa.

It is known, for example, that androgen receptor (AR), a classic steroid receptor which is known to activate transcription of AR-responsive genes, also affects the Wnt signaling pathway via the androgen-mediated interaction of AR with β-catenin (1,2,3). The Wnt pathway plays an important role in the differentiation and functional activity of a variety of tissues including bone, intestine, skin, and hair follicles. Alterations in the Wnt pathway have been implicated in disease states such as osteoporosis and prostate and colon cancer. Other conditions where Wnt signaling may become altered include insulin resistance in polycystic ovary syndrome and androgenic alopecia (4,5). In many of these conditions, androgens and AR have been implicated as being potential modulators of the Wnt pathway.

This invention describes a novel screening assay to identify compounds that modulate the interaction of androgen receptor with β-catenin. When used in conjunction with standard assays measuring modulation of classical androgen mediated AR-dependent transcription, the assay of the invention enables the user to identify new classes of AR modulators that selectively inhibit the ability of AR to interact with β-catenin and modulate its activity without affecting classical AR agonist or antagonist activity such as, for example, androgen-mediated transcription by AR. Compounds with this selective activity could not be detected using classical AR transcriptional assays alone.

### SUMMARY OF THE INVENTION

This invention provides a method of determining if a test compound is able to modulate the interaction between androgen receptor (AR) and β-catenin comprising the steps of:
(a) providing a cell comprising:
   (i) a DNA sequence encoding a hybrid protein comprising a DNA binding domain fused to the NH₃-terminal region of β-catenin,
   (ii) a DNA sequence comprising an upstream activation sequence corresponding to said DNA binding domain operably linked to and controlling transcription of a reporter gene, and
   (iii) a DNA sequence encoding androgen receptor protein,
(b) introducing the test compound to the cell, optionally in the presence of androgen; and
(c) measuring the expression of the reporter gene,
   wherein a decrease or increase of expression by the reporter gene indicates that the test molecule is able to modulate the interaction between androgen receptor and β-catenin.

In a preferred mode, this invention further provides a method wherein the DNA binding domain of (i) comprises a GAL-4 DNA binding domain; and the upstream activation DNA sequence operably linked to a reporter gene of (ii) comprises GAL-4 UAS operably linked to the reporter gene.

In a more preferred mode, this invention further provides wherein the NH₃-terminal region β-catenin of (i) comprises amino acids 2 through 424 of human β-catenin; wherein the reporter gene is luciferase; wherein there are multiple copies of the GAL-4 UAS sequence operably linked to the luciferase gene; wherein the modulation accomplished by the test compound is a decrease in the expression of the luciferase gene; and wherein the androgen at step (b) is DHT.

Another aspect of the invention is for a method of determining if a test compound is able to modulate the interaction between androgen receptor and β-catenin comprising the steps of:
(a) providing a cell comprising:
   (i) a DNA sequence encoding a hybrid protein comprising a DNA binding domain fused to β-catenin,
   (ii) a DNA sequence comprising an upstream activation sequence corresponding to said DNA binding domain operably linked to a reporter gene, and
   (iii) a DNA sequence encoding androgen receptor protein,
(b) introducing the test compound to the cell, optionally in the presence of androgen; and
(c) measuring the expression of the reporter gene, wherein an increase or decrease of expression by the reporter gene indicates that the test molecule is able to modulate the interaction between androgen receptor and β-catenin.

Another aspect is for a method of determining if a test compound selectively modulates the β-catenin-Wnt signaling pathway over an androgen receptor signaling pathway comprising:
(a) identifying a test compound which increases or decreases the expression of a gene by inhibiting the AR mediated interaction with β-catenin, wherein the test compound removes androgen-liganded AR repression on Wnt signaling without repressing androgen-AR mediated transcription; and
(b) assaying the test compound of (a) to determine whether the test compound increases or decreases the expression of a gene through a β-catenin independent androgen receptor signaling pathway;
   whereby the test compound of (a) selectively modulates the β-catenin-Wnt signaling pathway by inhibiting the ability androgen-liganded AR to interact with β-catenin if the test compound fails to increase or decrease the expression of a gene through an androgen receptor signaling pathway.

A further aspect is for a method of determining if a test compound selectively modulates an androgen receptor signaling pathway over a β-catenin-Wnt signaling pathway comprising:
(a) identifying a test compound which increases or decreases the expression of a gene through an androgen receptor signaling pathway; and

(b) assaying the test compound of (a) to determine whether the test compound increases or decreases the ability of androgen-liganded AR or non-liganded AR to inhibit β-catenin-Wnt signaling;

whereby the test compound of (a) selectively modulates an androgen receptor signaling pathway without removing androgen-liganded AR repression of Wnt signaling or does not promote the interaction between AR and β-catenin in the absence of an AR agonist resulting in the test compound having no activity in increasing or decreasing the expression of a gene regulated by β3-catenin-Wnt signaling pathway.

### BRIEF DESCRIPTION OF THE FIGURES

FIG. 1 depicts the plasmid constructs used in one embodiment of the invention.

FIG. 2 is a Western Blot demonstrating that dihydrotestosterone (DHT) stimulates the interaction between AR and β-catenin in L929 cells.

FIG. 3 is a bar graph illustrating that the method of the invention measures the DHT dependent interaction between AR and β-catenin.

FIG. 4 is a bar graph illustrating that the protein-protein interaction between β-catenin and nuclear receptors is specific for AR.

FIG. 5 is a graph illustrating that the DHT stimulated interaction between AR and β-catenin is inhibited by the AR antagonist cyproterone acetate.

FIG. 6 depicts the amino acid sequence for human β-catenin (GenBank® Accession # 2208332A; SEQ ID NO:1).

### DETAILED DESCRIPTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

This invention assesses the androgen dependent interaction between AR and β-catenin, and utilizes i) a first DNA sequence comprising DNA encoding a hybrid protein comprising a DNA binding domain fused to β-catenin, ii) a second DNA sequence comprising an upstream activation sequence able to recognize the DNA binding domain of (i) which is operably linked to a reporter gene; and iii) a third DNA sequence encoding AR protein. The method of the invention entails providing test compounds to a cell comprising and able to express the DNA sequences i, ii and iii, optionally in the presence of an androgen, to determine if the test compound is able to modulate the androgen-stimulated interaction of β-catenin and androgen receptor, as measured by detections of expression of the reporter gene. If expression of the reporter gene is unaffected by addition of the test compound to the cell, such compound is unable to modulate the androgen-dependent interaction of β-catenin with androgen receptor. In a preferred mode, the DNA binding domain of (i) comprises the DNA binding domain of GAL4; the upstream activation sequence operably linked to the reporter gene of (ii) is GAL4-UAS; and the reporter gene is luciferase. In a most preferred embodiment of the invention, multiple copies of the upstream activation sequence are operably linked to the reporter gene, and the β-catenin of (i) that is fused to the DNA binding domain comprises amino acids 2 to 424 of human β-catenin.

Applicants have demonstrated that their assay is a selective measure of the androgen-dependent interaction between AR and β-catenin. Generally, in screening mode, cells transformed with the DNA sequences of the invention are treated with an androgen such as dihydrotestosterone (DHT) which causes the interaction of AR and β-catenin resulting in increased reporter activity. Molecules that lower reporter activity can be identified and further tested in secondary assays for their ability to disrupt the interaction between AR and DNA response consensus elements which bind liganded AR and cause activation or inhibition of transcription. Molecules identified by this methodology in this screen may be particularly useful as treatments for androgenic alopecia, prostate cancer, and insulin sensitivity in polycystic ovary syndrome.

Applicants' assay is similar to the two-hybrid binding assay of Fields et al. (6,7), which utilizes one hybrid comprising a protein fused to a DNA binding domain, and a second hybrid comprising a protein fused to a transcription activating domain. The current one-hybrid binding assay is distinct, however, in that it should favor the identification of molecules that may be able to disrupt the interaction of AR and β-catenin and thereby specifically remove the effect of β-catenin on AR signaling or remove the effect of AR on β-catenin and Wnt signaling. Further, in a preferred mode, only a portion of the β-catenin coding sequence is fused to the GAL-4 DNA binding domain (GAL-4 DBD). Because the present assay utilizes full length, wild type AR, the entire AR protein is available for targeting and test compounds are not blocked from binding to AR by a fusion construct. In contrast, in the two-hybrid assay (6,7), the use of two recombinant fusion protein constructs has the disadvantage that the natural conformation of the target protein may be altered in the fusion construct.

An assay of the invention may be conducted in, for example, the well-characterized and widely used CV-1 cells (African green monkey kidney cell line) or COS cells (African green monkey kidney cell line), but one of ordinary skill in the art would recognize that other cell lines are suitable as well.

A further aspect of the invention is for a method of determining if a test compound selectively modulates a β-catenin-Wnt signaling pathway over an androgen receptor signaling pathway. In the method, test compounds are identified based on their ability to increase or decrease the expression of a gene by modulating the androgen-AR mediated repression of the β-catenin-Wnt signaling pathway. “Androgen liganded androgen receptor-mediated modulation of the β-catenin-Wnt signaling pathway” or, as used herein, refers to a signaling pathway resulting in a transcriptional increase or decrease of any gene modulated by the Wnt-mediated signaling pathway initiated through an androgen receptor/β-catenin interaction (see, e.g., Mulholland D J et al., J. Biol. Chem. 277:17933-43 (2002); Yang F. et al., J. Biol. Chem. 277:1133644 (2002); Song L-N et al., Mol. Cell. Biol. 23:1674-87 (2003)).

Methods of assessing the interaction of androgen receptor with β-catenin are preferentially utilized to identify test compounds capable of increasing or decreasing the expression of a gene by modulating the interaction of AR and β-catenin resulting in repression or inhibition of androgen-AR mediated repression on the β-catenin-Wnt signaling pathway. In a cell type specific context, the interaction between AR and β-catenin may have a positive effect on Wnt signaling or AR mediated signaling. In this embodiment, positive regulators of the AR-β-catenin interaction would be developed as Wnt activators.

A test compound that positively or negatively affects the ability of androgen-liganded AR to modulate β-catenin-Wnt transcriptional signaling is then assayed to determine whether the test compound increases or decreases the expression of a gene through an androgen receptor signaling pathway. “Androgen receptor signaling pathway” or “AR signaling pathway”, as used herein, refers to the traditional transcriptional pathway of the androgen receptor. In response to a ligand binding, androgen receptor migrates to the nucleus of a cell where it forms a homodimer. Upon binding to an androgen response element (ARE) as a homodimer, agonist-bound AR stimulates transcription by recruiting a large enzymatic co-activator complex that includes GRIP1/TIF2, CBP/p300, and other coactivators. In addition, ligand-bound AR can also suppress transcription via protein-protein interaction with transcription factor complexes such as, for example, AP1, NF-κB, and Ets family.

One of ordinary skill in the art would recognize that any assay of AR signaling pathway assessment is useful in the present invention.

Test compounds that fail to increase or decrease the expression of a gene through an androgen signaling pathway, in the presence or absence of natural endogenous or exogenous androgens, selectively modulate a β-catenin-Wnt signaling pathway. By “fails to increase or decrease the expression of a gene” is meant that no increase or decrease of gene expression is observable through assaying techniques known to one of ordinary skill in the art such as, for example, Northern Blotting, Western Blotting, Southern Blotting, plasmid reporter assays, or polymerase chain reaction (PCR), or by observing overall changes in in vivo (animal) organ system morphology or functions known to be modulated by androgens or β-catenin. An example of known animal model effects of androgens would be the effects of AR modulators and Wnt modulators on prostate growth/weight in rodents/mammals where AR agonists increase prostate cell growth and organ weight and AR antagonists inhibit this effect of androgens.

An alternate embodiment is for a method determining if a test compound selectively modulates an androgen receptor signaling pathway over a β-catenin-Wnt signaling pathway. In the method, test compounds are identified based on their ability to increase or decrease the expression of a gene through an androgen receptor signaling pathway by methods as are well known to those of ordinary skill in the art. A test compound that positively or negatively affects AR signaling is then assayed to determine whether the test compound increases or decreases the expression of a gene through an AR-mediated β-catenin-Wnt signaling pathway as described above.

Within the context of Applicants' disclosure, terms will have their customary technical meaning in the art unless otherwise stated. Some terms and aspects of the invention are further described below.

The term “androgen receptor” or “AR” refers to the AR protein as defined by its conserved amino acid coding sequence in an active or native structural conformation.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. Under stringent conditions, nucleic acid molecules at least 65%, 70%, 75% or more identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide if hybridization can occur between one of the strands of the first polynucleotide and the second. “Complementarity” or homology is quantifiable in terms of the proportion of bases in opposing strands that are expected to hydrogen bond with each other, according to generally accepted base-pairing rules.

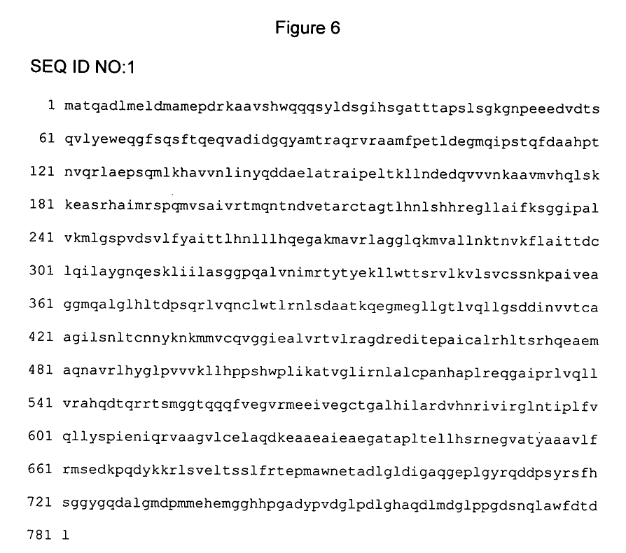

The term “β-catenin” is used to encompass full-length proteins comprising, for example, in the human protein, 781 amino acids and also fragments of the β-catenin amino acid sequence as disclosed, for example, in FIG. 6 (SEQ ID NO:1). Preferred forms of the protein include particularly amino acids about 1 through about 423. In other embodiments, a β-catenin protein has at least 65%, at least 70% amino acid identity, more preferably 80% amino acid identity, more preferably 90%, and even more preferably, 95% amino acid identity with the amino acid sequence shown in SEQ ID NO:1 or a portion thereof.

In another embodiment, the term “β-catenin” is used to encompass full-length proteins or fragments thereof encoded by polynucleotides which hybridize under stringent conditions to a polynucleotide encoding the amino acid sequence of SEQ ID NO:1, or a fragment or complement thereof. Preferably, the conditions are such that sequences at least 65%, preferably at least about 70%, more preferably at least about 80%, even more preferably at least about 85% or 90% homologous to each other typically remain hybridized to each other. Preferably, a β-catenin polynucleotide that hybridizes under stringent conditions to a polynucleotide sequence which encodes the amino acid sequence of SEQ ID NO: 1 or fragments or complements thereof corresponds to a naturally-occurring nucleic acid molecule.

In addition to naturally-occurring allelic variants of β-catenin sequences that may exist in the population, the skilled artisan will further appreciate that minor changes may be introduced by mutation into polynucleotide sequences which encode, for example, the amino acid sequence of SEQ ID NO:1, thereby leading to changes in the amino acid sequence of the encoded protein, without altering the functional activity of a β-catenin protein. For example, nucleotide substitutions leading to amino acid substitutions at “non-essential” amino acid residues may be made in a polynucleotide sequence which encodes the amino acid sequence of SEQ ID NO:1. A “non-essential” amino acid residue is a residue that can be altered from the wild-type sequence of a β-catenin polynucleotide (e.g., a polynucleotide encoding the amino acid sequence of SEQ ID NO:1) without altering the functional activity of a β-catenin molecule. Exemplary residues which are non-essential and, therefore, amenable to substitution can be identified by one of ordinary skill in the art by performing an amino acid alignment of β-catenin-related molecules and determining residues that are not conserved. Such residues, because they have not been conserved, are more likely amenable to substitution.

Accordingly, the term “β-catenin” also pertains to polynucleotides encoding β-catenin proteins that contain changes in amino acid residues that are not essential for a β-catenin activity. Such β-catenin proteins differ in amino acid sequence of SEQ ID NO:1 yet retain an inherent β-catenin activity. An isolated polynucleotide encoding a non-natural variant of a β-catenin protein can be created by introducing one or more nucleotide substitutions, additions, or deletions into a polynucleotide sequence encoding the amino acid sequence of SEQ ID NO:1 such that one or more amino acid substitutions, additions, or deletions are introduced into the encoded protein. Mutations can be introduced into SEQ ID NO:1 by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a β-catenin polypeptide is preferably replaced with another amino acid residue from the same side chain family.

Alternatively, in another embodiment, mutations can be introduced randomly along all or part of a β-catenin coding sequence, such as by saturation mutagenesis.

The term “NH₃-terminal region of β-catenin” comprises any contiguous amino acid sequence from amino acid one through the armadillo repeat regions of β-catenin capable of interacting with androgen receptor. Thus, the NH₃-terminal region can comprise, for example, amino acid 1 through amino acid 424, amino acid 2 through amino acid 424, amino acid 3 through amino acid 424, amino acid 1 through amino acid 423, amino acid 2 through 423, amino acid 3 through 423, and so forth. The NH₃-terminal region preferably comprises armadillo repeats 1-6 of β-catenin, more preferably armadillo repeats 1-7 of β-catenin, and even more preferably armadillo repeats 1-12 of β-catenin. In another preferred embodiment, the NH₃-terminal region is amino acids 2-424 of human β-catenin. The NH₃-terminal region can comprise amino acid sequences from only the armadillo repeat region. Embodiments comprising an NH₃-terminal region amino acid sequence contiguous with at least a portion of the C-terminal region of β-catenin are also contemplated so long as the C-terminal trans-activation domain of β-catenin is inactive. Conservative substitutions, deletions, or insertions of amino acids are also contemplated so long as an interaction between β-catenin and androgen receptor is maintained.

Typical substitutions include, for example, substitution of an amino acid with an amino acid having similar charge, hydrophobic, or stereochemical characteristics. For example, a “conservative amino acid substitution” may involve a substitution of a native amino acid residue with a normative residue such that there is little or no effect on the polarity or charge of the amino add residue at that position. Desired amino acid substitutions (whether conservative or non-conservative) can be determined by those skilled in the art at the time such substitutions are desired. For example, amino acid substitutions can be used to identify important residues of the molecule sequence, or to increase or decrease the affinity of the molecules described herein. In certain embodiments, conservative amino acid substitutions also encompass non-naturally occurring amino acid residues which are typically incorporated by chemical peptide synthesis rather than by synthesis in biological systems.

The term “interacting with androgen receptor” means the protein-protein interaction between the ligand binding domain of AR and the armadillo regions of β-catenin. The interaction between these two proteins is caused by changes in the AR protein secondary/tertiary conformation which is stimulated by androgen binding to AR.

The term “modulate” encompasses either a decrease or an increase in activity; for example, a test compound can be considered to modulate the interaction between androgen receptor and β-catenin if the presence of such test compounds in the assay of the invention results in either a decrease or increase in the expression of luciferase gene.

The term “DNA binding domain” describes any protein binding domain that has a conserved DNA binding motif that binds in a sequence specific manner to its conserved upstream activation sequence also referred to as a “DNA response element” that contains the specific nucleotide sequence or “recognition sequence” that is recognized by the protein DNA binding domain. The DNA response element is placed in a reporter plasmid so that proteins that bind to the DNA response element are capable of bringing transcriptional activators in close proximity to the reporter through protein-protein interactions resulting in activation of reporter transcription.

The term “upstream activation sequence” or “UAS” includes any DNA sequence which is able to bind the DNA binding domain which has been selected for use in the assay as a fusion protein with β-catenin.

The term “reporter gene” is used in the manner commonly known in the art to describe any genetic coding sequence which is able to express a protein or amino acid sequence that can be detected and quantitated. Examples of well known reporter gene productions that could be used in the assay of the invention include, for example, the enzymes luciferase, chloramphenicol actyltransferase, and β-galactosidase. Those skilled in the art will know many other suitable reporter genes.

The term “test compound” includes compounds with known chemical structure but not necessarily with a known function or biological activity. Test compounds could also have unidentified structures or be mixtures of unknown compounds, for example from crude biological samples such as plant extracts. Large numbers of compounds could be randomly screened from “chemical libraries” which refers to collections of purified chemical compounds or collections of crude extracts from various sources. The chemical libraries may contain compounds that were chemically synthesized or purified from natural products. The compounds may comprise inorganic or organic small molecules or larger organic compounds such as, for example, proteins, peptides, glycoproteins, steroids, lipids, phospholipids, nucleic acids, and lipoproteins. The amount of compound tested can very depending on the chemical library, but, for purified (homogeneous) compound libraries, 10 μM is typically the highest initial dose tested.

Methods of introducing test compounds to cells are well known in the art.

The term “androgen” includes all known compounds with androgenic activity. Androgenic activity of compounds may be determined in a variety of ways including in cell-based AR transcription assays and in biological activity assays where a compound can be demonstrated to have activity that is similar to the activity of known androgens. These assays can be performed using animals or tissues. For example, compounds with androgen activity in the prostate are able to stimulate prostate growth in rodents. Natural androgen metabolites that have biological activity can be used and include, for example, testosterone, androstenedione, androstanedione, and dihydrotestosterone (DHT), with DHT particularly preferred.

The assay is tolerant of a wide concentration range of androgens. In a preferred mode, the DHT dose is 1 nM to screen compounds. Between 0.1 and 10 nM of androgen could be used as an initial dose to optimize the assay in a cell line.

In the absence of androgen, an assay of the present invention can also be used to identify compounds that stimulate the interaction between AR and β-catenin. For example, a compound with activity similar to DHT would activate reporter activity through the stimulation of the interaction between AR and β-catenin.

The term “operably linked” means that a nucleic acid molecule, i.e., DNA, and one or more regulatory sequences (e.g., a promoter or portion thereof) are connected in such a way as to permit transcription of mRNA from the nucleic acid molecule or permit expression of the product (i.e., a polypeptide) of the nucleic acid molecule when the appropriate molecules are bound to the regulatory sequences.

The term “expression construct” means any double-stranded DNA or double-stranded RNA designed to transcribe an RNA, e.g., a construct that contains at lease one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus, or further embodiments described under “expression vector” below. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct. Further, the term expression construct or vector is intended to also include instances wherein the cell utilized for the assay already endogenously comprises such DNA sequence.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A), cytosine (C), guanine (G), thymine (T), and uracil (U) for guanine when the polynucleotide is RNA. Thus, the term “polynucleotide sequence” is the alphabetical representation of a polynucleotide molecule.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T., Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase 11, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

### Example 1

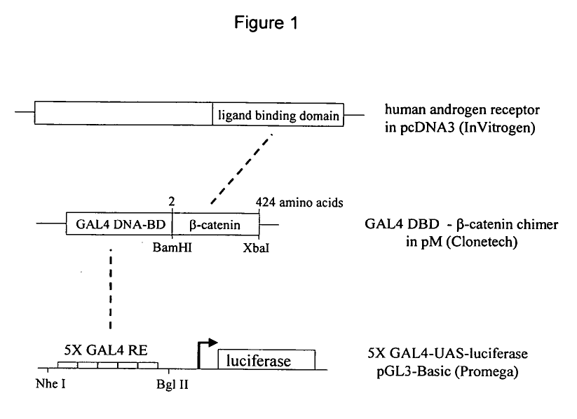

This Example illustrates a preferred embodiment wherein cultured cells were transformed with a GAL4 DNA response element-luciferase reporter plasmid, a plasmid expressing the coding region of the GAL4 DBD fused to the cDNA coding region for amino acids 2-424 of human β-catenin (GAL4-β-catenin) and a plasmid expressing full length wild type human AR (FIG. 1). The β-catenin cDNA fragment was made by PCR amplification of the DNA coding sequence for amino acids 2-424 from human β-catenin using a pcDNA3.1 β-catenin expression vector (Invitrogen) as a template and single stranded DNA primers containing Bam HI and Xba I restriction sites respectively. The amplified DNA fragment was inserted into the multiple cloning site of the pM plasmid which was linearized using the restriction enzymes Bam HI and Xba I (Promega) and which contains the coding sequence for the GAL4 DBD upstream of the multiple cloning site. The reporter plasmid was made by sub-cloning five copies of the GAL4 upstream activation sequence (UAS) into pGL3-Basic (Promega) which contains the cDNA coding sequence for luciferase. The AR expression vector is human full length AR cDNA in pcDNA3 (Invitrogen) that was obtained from Leonard Freedman (Sloan Kettering). The cDNA sequence for human AR is available on the gene sequence information website for GenBank® (Accession No. M35884, incorporated herein by reference).

CV-1 cells were cultured on 96 well plates and transfected after 24 hours with the expression and reporter plasmids using the lipofectamine procedure (Invitrogen). In this procedure, the three different plasmid constructs used were mixed in cell culture media and lipofectamine and incubated for 15 minutes according to the manufacturer's instructions (Invitrogen). The plasmid-lipofectamine mixture was diluted in culture medium, added to cells, and incubated for 4 hours. The cells were rinsed and then treated with culture media. Twenty-four hours after transfection, the cells were treated with androgen agonists and antagonists or vehicle. After 18 hours, cell lysates were harvested and assayed for luciferase activity using luciferase reagent (Promega) and a luminometer (Wallac). Other known transfection techniques can be used including, for example, calcium phosphate precipitation transfection technique.

The plasmid constructs used in the one-hybrid assay are shown in FIG. 1. Dotted lines denote known protein-protein or protein-DNA interaction regions employed in the one-hybrid assay. Arrows denote transcription start sites.

### Example 2

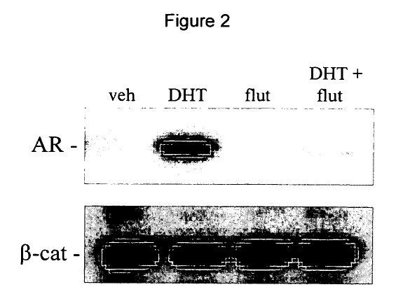

L929 cells were used to determine if DHT stimulates the interaction between AR and β-catenin in a cell line that endogenously expresses both proteins. L929 cells, which express endogenous AR and β-catenin, were treated with 10 nM DHT, 300 nM hydroxyflutamide (flut), DHT plus flut, or vehicle (veh) for 17 hours. Cell lysates were harvested, precleared with protein A/G sepharose, and β-catenin was immunoprecipitated using a goat polyclonal IgG against β-catenin conjugated to agarose (Santa Cruz Biotechnology). The immunoprecipitates were analyzed by polyacrylamide gel electrophoresis followed by Western analysis for AR and β-catenin (β-cat) as indicated using antibodies from Santa Cruz (FIG. 2). The androgen agonist DHT at 10 nM was found to stimulate the interaction between AR and β-catenin. There was no detectable interaction between these proteins in the absence of DHT. When cells were treated with DHT in the presence of a 30-fold excess of the AR antagonist hydroxyflutamide. (300 nM) over DHT, the protein-protein interaction was attenuated (FIG. 2). These results demonstrate that the AR and β-catenin interaction is stimulated by the androgen agonist DHT. The results also demonstrate that this interaction is susceptible to disruption by small molecules such as hydroxyflutamide.

### Example 3

Experiments were performed to determine the requirement of each expression vector for luciferase activity in CV-1 cells.

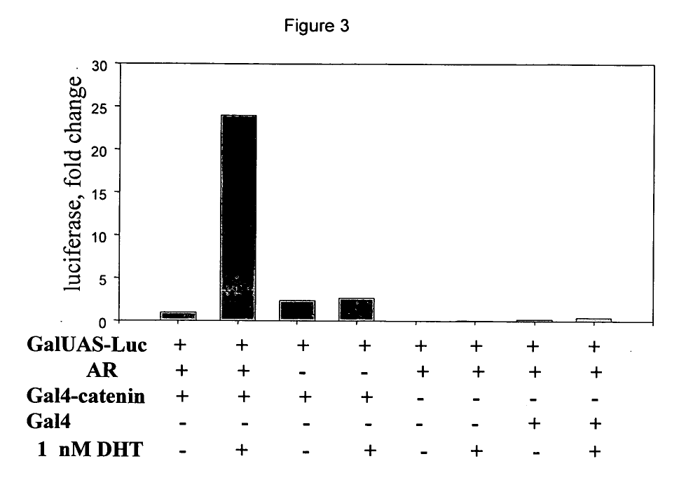

The assay of the invention is demonstrated to measure the DHT dependent interaction between AR and β-catenin. The reporter plasmid (GAL4-luciferase) containing the luciferase gene under transcriptional control of the 5XGAL4-UAS DNA response element was transfected into CV-1 cells in the presence or absence of the androgen receptor expression vector (AR), the GAL4-DBD-β-catenin fusion protein expression vector (GAL4-β-catenin) as indicated. Cells were treated with 1 nM DHT (+) or vehicle (−) for 18 hours where indicated. Cell lysates were harvested and analyzed for luciferase activity. DHT (1 nM) caused a large activation of reporter activity when both the AR and GAL4-β-catenin expression vectors were transfected into the cells with the 5XGAL4-luciferase reporter (FIG. 3). AR and DHT had no effect on reporter activity in the absence of the GAL4-β-catenin expression vector demonstrating the absence of a direct effect of AR and DHT on the GAL4 promoter-reporter construct (FIG. 3).

### Example 4

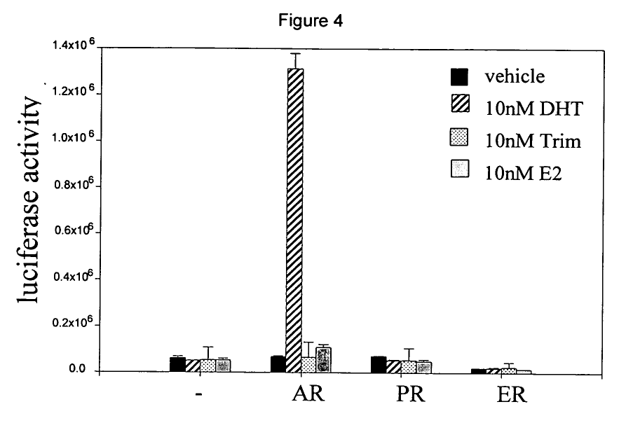

The estrogen receptor (ER) and progesterone receptor (PR) were also tested for their ability to interact with β-catenin by measuring their effect on reporter activity (FIG. 4), and the protein-protein interaction between β-catenin and nuclear receptors was shown to be specific for AR. CV-1 cells were transfected with the 5XGAL4-luciferase reporter and the GAL4-O-catenin expression plasmid in the absence of a nuclear receptor expression vector (−), with the AR, PR, or ER expression plasmid. The cells were treated for 18 hours with vehicle, 10 nm DHT, 10 nM trimegestone (Trim), or 10 nM 17β-estradiol (E2) as indicated. Cell lysates were harvested and analyzed for luciferase activity. The ER and PR receptors had no activity in the one-hybrid assay in the presence of trimegestone, 17β-estradiol, or DHT. There was a large increase in reporter activity when DHT was added to AR expressing cells but not with 10 nM 17β-estradiol or trimegestone. These results demonstrate that the interaction of β-catenin in this assay with nuclear receptors appears to be specific for AR when compared to ER and PR.

### Example 5

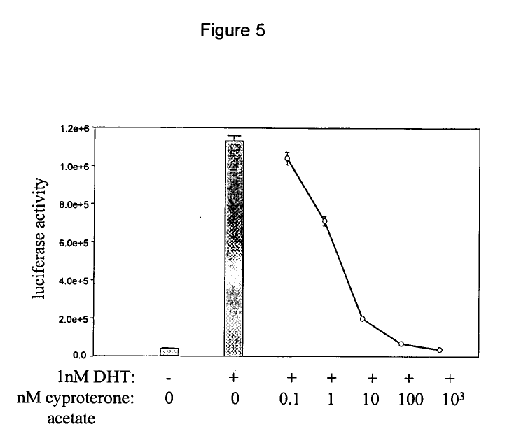

To determine if Applicants' assay has the potential to identify compounds that disrupt the DHT-dependent interaction of AR and β-catenin, the effect of cyproterone acetate (CA), an AR antagonist, was tested in the presence of DHT (FIG. 5).

CV-1 cells were transfected with the AR and GAL4-β-catenin expression and luciferase reporter plasmids described in FIG. 1. Cells were treated for 18 hours with (+) or without (−) 1 nM DHT and the indicated concentrations of the AR antagonist cyproterone acetate. Cell lysates were harvested and analyzed for luciferase activity. The DHT stimulated interaction between AR and β-catenin is inhibited by the AR antagonist cyproterone acetate. In the absence of CA, DHT at 1 nM caused a large activation of reporter activity. This effect of DHT was dose dependently reversed by CA from between 1 and 1000 nM (FIG. 5).

### REFERENCES

1. Yang F, Li X, Sharma M, Sasaki C, Longo D, Lim B, Sun Z. (2002) J. Biol. Chem. 277:13; 11336-11344.
2. Pawlowski J, Ertel J, Allen M, Xu M, Butler C, Wilson E, Wierman M. (2002) J. Biol. Chem. 277: 23; 20702-20710.
3. Song L, Herrell R, Byers S, Shah S, Wilson E, Gelmann E. (2003) Mol. Cell. Biol. 23: 5; 1674-1687.
4. Sharma M, Chuang W, Sun Z (2002) J. Biol. Chem. 277: 34; 30935-30941.
5. Truica C, Byers S, Gelmann E. (2000) Cancer Res. 60: 4709-4713.
6. U.S. Pat. No. 5,283,173, Fields, et al., “System to Detect Protein-Protein Interactions”.
7. U.S. Pat. No. 5,468,614, Fields, et al., “System to Detect Protein-Protein Interactions”.

What is claimed is:

1. A method of determining if a test compound is able to modulate the interaction between androgen receptor and β-catenin comprising the steps of:
(a) providing a cell comprising:
(i) a DNA sequence encoding a hybrid protein comprising a DNA binding domain fused to the NH₃-terminal region of β-catenin,
(ii) a DNA sequence comprising an upstream activation sequence corresponding to said DNA binding domain operably linked to and controlling transcription of a reporter gene, and
(iii) a DNA sequence encoding androgen receptor protein,
(b) introducing the test compound to the cell, optionally in the presence of androgen; and
(c) measuring the expression of the reporter gene,
wherein an increase or decrease of expression by the reporter gene indicates that the test molecule is able to modulate the interaction between androgen receptor and β-catenin.

2. The method of claim 1, wherein the DNA binding domain of (i) comprises a GAL-4 DNA binding domain.

3. The method of claim 1, wherein the upstream activation sequence operably linked to a reporter gene of (ii) comprises GALA UAS.

4. The method of claim 3, wherein there are one or more copies of the GALA UAS sequence operably linked to the reporter gene.

5. The method of claim 1, wherein the reporter gene is luciferase.

6. The method of claim 1, wherein the NH₃-terminal region of β-catenin of (i) comprises amino acids 2 through 424 of human β-catenin.

7. The method of claim 1, wherein the NH₃-terminal region of β-catenin of (i) comprises a nucleotide sequence encoding an amino acid sequence having at least 65% identity with amino acids 2424 of SEQ ID NO:1.

8. The method of claim 7, wherein the NH₃-terminal region of β-catenin of (i) comprises a nucleotide sequence encoding an amino acid sequence having at least 75% identity with amino acids 2-424 of SEQ ID NO:1.

9. The method of claim 8, wherein the NH₃-terminal region of β-catenin of (i) comprises a nucleotide sequence encoding an amino acid sequence having at least 85% identity with amino acids 2424 of SEQ ID NO:1.

10. The method of claim 9, wherein the NH₃-terminal region of β-catenin of (i) comprises a nucleotide sequence encoding an amino acid sequence having at least 95% identity with amino acids 2424 of SEQ ID NO:1.

11. The method of claim 1, wherein the NH₃-terminal region of β-catenin of (i) comprises a nucleotide sequence which hybridizes with a nucleotide sequence encoding amino acids 2424 of SEQ ID NO:1 under the following conditions: 6×SSC at 45° C. and washed at least once with 0.2×SSC, 0.1% SDS at 50° C.

12. The method of claim 11, wherein the NH₃-terminal region of β-catenin of (i) comprises a nucleotide sequence which hybridizes with a nucleotide sequence encoding amino acids 2424 of SEQ ID NO:1 under the following conditions: 6×SSC at 45° C. and washed at least once with 0.2×SSC, 0.1% SDS at 55° C.

13. The method of claim 12, wherein the NH₃-terminal region of β-catenin of (i) comprises a nucleotide sequence which hybridizes with a nucleotide sequence encoding amino acids 2-424 of SEQ ID NO:1 under the following conditions: 6×SSC at 45° C. and washed at least once with 0.2×SSC, 0.1% SDS at 65° C.

14. The method of claim 1, wherein said cell is a eukaryotic cell.

15. The method of claim 14, wherein said cell is a mammalian cell.

16. The method of claim 1, wherein the modulation of expression of the reporter gene is a decrease in expression.

17. The method of claim 1, wherein said androgen at step (b) is DHT.

18. The method of claim 1, wherein the DNA binding domain of (i) comprises a GAL-4 DNA binding domain; wherein the NH₃-terminal region of β-catenin of (i) comprises amino acids 2 through 424 of human β-catenin; wherein there are more than one copy of the upstream activation sequence GAL-4 UAS operably linked to the reporter gene; wherein the reporter gene is luciferase; wherein the androgen at step (b) is DHT; and
wherein the modulation is a decrease in expression.

19. The method of claim 1, wherein the NH₃-terminal region of β-catenin comprises armadillo repeats 1-12.

20. The method of claim 1, wherein the NH₃-terminal region of β-catenin comprises armadillo repeats 1-7.

21. The method of claim 1, wherein the NH₃-terminal region of β-catenin comprises armadillo repeats 1-6.

22. A method of determining if a test compound is able to modulate the interaction between androgen receptor and β-catenin comprising the steps of:
(a) providing a cell comprising:
(i) a DNA sequence encoding a hybrid protein comprising a DNA binding domain fused to β-catenin,
(ii) a DNA sequence comprising an upstream activation sequence corresponding to said DNA binding domain operably linked to and controlling transcription of a reporter gene, and
(iii) a DNA sequence encoding androgen receptor protein,
(b) introducing the test compound to the cell, optionally in the presence of androgen; and
(c) measuring the expression of the reporter gene,
wherein an increase or decrease of expression by the reporter gene indicates that the test molecule is able to modulate the interaction between androgen receptor and β-catenin.

23. A method of determining if a test compound selectively modulates the β-catenin-Wnt signaling pathway over an androgen receptor signaling pathway comprising:
(a) identifying a test compound which increases or decreases the expression of a gene by inhibiting the AR mediated interaction with β-catenin, wherein the test compound removes androgen-liganded AR repression on Wnt signaling without repressing androgen-AR mediated transcription; and
(b) assaying the test compound of (a) to determine whether the test compound increases or decreases the expression of a gene through a β-catenin independent androgen receptor signaling pathway;
whereby the test compound of (a) selectively modulates the β-catenin-Wnt signaling pathway by inhibiting the ability androgen-liganded AR to interact with β-catenin if the test compound fails to increase or decrease the expression of a gene through an androgen receptor signaling pathway.

24. The method of claim 23, wherein step (a) comprises the steps of
(A) providing a cell comprising:
(i) a DNA sequence encoding a hybrid protein comprising a DNA binding domain fused to β-catenin;
(ii) a DNA sequence comprising an upstream activation sequence corresponding to said DNA binding domain operably linked to and controlling transcription of a reporter gene; and
(iii) a DNA sequence encoding androgen receptor protein;
(B) introducing the test compound to the cell, optionally in the presence of androgen; and
(C) measuring the expression of the reporter gene,
wherein an increase or decrease of expression by the reporter gene indicates that the test molecule is able to modulate the interaction between androgen receptor and β-catenin.

25. The method of claim 24, wherein the DNA sequence of (i) encodes a hybrid protein comprising a DNA binding domain fused to the NH₃-terminal region of β-catenin.

26. A method of determining if a test compound selectively modulates an androgen receptor signaling pathway over a β-catenin-Wnt signaling pathway comprising:
(a) identifying a test compound which increases or decreases the expression of a gene through an androgen receptor signaling pathway; and
(b) assaying the test compound of (a) to determine whether the test compound increases or decreases the ability of androgen-liganded AR or non-liganded AR to inhibit β-catenin/Wnt signaling;
whereby the test compound of (a) selectively modulates an androgen receptor signaling pathway without removing androgen-liganded AR repression of Wnt signaling or does not promote the interaction between AR and β-catenin in the absence of an AR agonist resulting in the test compound having no activity in increasing or decreasing the expression of a gene regulated by β-catenin-Wnt signaling pathway.

27. The method of claim 26, wherein step (b) comprises the steps of
(A) providing a cell comprising:
(i) a DNA sequence encoding a hybrid protein comprising a DNA binding domain fused to β-catenin;
(ii) a DNA sequence comprising an upstream activation sequence corresponding to said DNA binding domain operably linked to and controlling transcription of a reporter gene; and
(iv) a DNA sequence encoding androgen receptor protein;
(B) introducing the test compound of (a) to the cell, optionally in the presence of androgen; and
(C) measuring the expression of the reporter gene,
wherein an increase or decrease of expression by the reporter gene indicates that the test molecule is able to modulate the interaction between androgen receptor and β-catenin.

28. The method of claim 27, wherein the DNA sequence of (i) encodes a hybrid protein comprising a DNA binding domain fused to the NH₃-terminal region of β-catenin.


SEQUENCE LISTING (1 sequence)

SEQ ID NO: 1 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL